(19) United States
(12) Patent Application Publication
(10) Pub. No.: US 2026/0201338 A1
(43) Pub. Date: Jul. 16, 2026

(54) METHOD OF INDUCING UMBILICAL CORD MESENCHYMAL STEM CELLS TO DIFFERENTIATE INSULIN-LIKE CELLS AND AN APPLICATION THEREOF

(71) Applicant: Fangfang Wan, Hangzhou (CN)

(72) Inventor: Xiaodong Hu, Shijiazhuang (CN)

(21) Appl. No.: 19/058,488

(22) Filed: Feb. 20, 2025

(30) Foreign Application Priority Data
Jan. 14, 2025 (CN) 2025100558872

(51) Int. Cl. C12N 5/071; A61K 35/39; A61P 3/10

(52) U.S. Cl. CPC C12N 5/0676; A61K 35/39; A61P 3/10; C12N 2500/34; C12N 2500/38; C12N 2500/84; C12N 2501/105; C12N 2501/11; C12N 2501/115; C12N 2506/1369

(57) ABSTRACT

A method of inducing umbilical cord mesenchymal stem cells to differentiate insulin-like cells and an application thereof, the invention carries out reasonable culture of purified umbilical cord mesenchymal stem cells by adopting culture medium having different substances, and can efficiently and stably induce them to differentiate into insulin-like cells, and the percentage of insulin-positive cells can reach 80%. The cell preparation prepared after induction of umbilical cord mesenchymal stem cells of the invention can be used for the treatment of clinical diabetic patients with high therapeutic efficacy.

### 1. TECHNICAL FIELD

The invention relates to the technical field of stem cells, in particular to a method of inducing umbilical cord mesenchymal stem cells to differentiate insulin-like cells and an application thereof.

### 2. BACKGROUND ART

Umbilical cord Mesenchymal Stem Cells (mesenchymal stem cells) are multifunctional stem cells present in the umbilical cord tissue of newborns, which can differentiate into many kinds of tissue cells and have a broad clinical application prospect. Umbilical cord mesenchymal stem cells (mesenchymal stem cells) have high differentiation potential, and can be differentiated into osteoblasts, neuronal cells, insulin-like cells, etc. under certain conditions. It has broad clinical application prospects in tissue engineering of bone, cartilage, muscle, tendon, ligament, nerve, liver, endothelium and myocardium. And diabetes mellitus is the third most serious chronic non-communicable disease threatening human health after tumor and vascular lesions, with high lethality, high disability and high medical expenses. Insulin is a hormone secreted by pancreatic β-cells in the human pancreas, which is essential for regulating blood glucose. For example, patients with type I diabetes mellitus (T1D) are unable to secrete insulin on their own due to loss of pancreatic β cells caused by autoimmune reactions, and thus need to rely on exogenous insulin injections to maintain blood glucose homeostasis. Differentiation of stem cells to generate insulin-secreting cells may provide a new way and direction for diabetes treatment. Although the prior art, such as the Chinese invention “Method of inducing differentiation of umbilical cord mesenchymal stem cells to insulin-secreting-like cells” with application No. 2016109796896, which relies on a single culture medium in the process of inducing differentiation of umbilical cord mesenchymal stem cells to insulin-secreting-like cells, the efficiency of the induced differentiation is low, and the rate of positive expression of insulin cells (according to the markers) is less than 25%.

### 3. SUMMARY OF THE INVENTION

Therefore, based on the above background, the invention provides a method of inducing umbilical cord mesenchymal stem cells to differentiate insulin-like cells and an application thereof, with a high rate of insulin cell positivity, which may provide a new way and direction for the treatment of diabetes.

The technical solution of the invention is:
a method of inducing umbilical cord mesenchymal stem cells to differentiate insulin-like cells, comprising the following steps:
S1: preparing and obtaining umbilical cord mesenchymal stem cells from umbilical cord;
S2: taking purified umbilical cord mesenchymal stem cells and add them to an α-MEM culture solution containing 10 ng/ml bFGF, 10 ng/ml EGF, and 5 wt % human platelet lysate, inducing differentiation of umbilical cord mesenchymal stem cells to nestin-positive progenitor cells NIP;
S3: digesting the cells after the cells are fully spread in the culture bottle, adding an α-MEM culture solution containing glucose and 5 wt % human platelet lysate, and culturing for 2-4 days;
S4: centrifugation, discard the culture supernatant, take cells, wash them, add them to an α-MEM culture solution containing ITS-A and 5 wt % human platelet lysate, culture for 3-5 days;
S5: centrifugation, discard culture supernatant, take cells, add them to an α-MEM culture solution containing 5 wt % human platelet lysate having 1 wt % ITS-A and nicotinamide, and culture for 2-4 days without changing the fluid during the period;
S6: take cells, discard culture supernatant, add them to an α-MEM culture solution containing 5 wt % human platelet lysate having 30 ng/ml β-cell regulator, 10 ng/ml IGF, 1 wt % ITS-A and nicotinamide, and culture for 2-5 days without changing the fluid during the period.

Further, concentration of glucose in the α-MEM culture solution in step S3 is 25 mM.

Further, concentration of ITS-A in the α-MEM culture solution in steps S4 to S6 is 1 wt %.

Further, concentration of nicotinamide in the α-MEM culture solution in each of steps S5 and S6 is 10 mM.

Further, conditions of cell culture in steps S3 to S6 are all: temperature: 37° C.-38° C., 5% CO₂.

Further, preparation of umbilical cord mesenchymal stem cells in step S1 comprises the following steps:
S1.1 taking umbilical cord, removing arteries and veins, cutting it into small segments, affixing it to a culture dish, and adding a special medium for umbilical cord mesenchymal stem cells dropwise to the small segments of umbilical cord;
S1.2 after incubation for 2-6 hours, replenish the special medium for umbilical cord mesenchymal stem cells;
S1.3 after continuing to culture for 5-7 days, digest and passage by using trypsin, and replenish the special medium for umbilical cord mesenchymal stem cells in the process of culture;
S1.4 when the cell density around tissue block reaches about 90%, trypsin digest cells, count them and transfer them for culture, and label them as P1 generation cells;
S1.5 perform cells passage until a 3rd generation, digest and collect the cells;
S1.6 purify and expand the 3rd generation cells collected at step S1.5 in α-MEM solution containing 5 wt % human platelet lysate.

Further, the special medium for umbilical cord mesenchymal stem cells of steps S1.1, S1.2, and S1.3 is α-MEM solution containing 5 wt % human platelet lysate, 100 U/ml penicillin, and 100 U/ml streptomycin.

Based on the same inventive concept, the invention also provides a cell preparation prepared from cells induced by the method for inducing the umbilical cord mesenchymal stem cells to differentiate insulin-like cells.

Based on the same inventive concept, the invention also provides a preparation method of the cell preparation, which is characterized by comprising the following steps: 1) taking cells induced by the method for inducing the umbilical cord mesenchymal stem cells to differentiate insulin-like cells;
2) centrifuging the induced cells after trypsin digestion using PBS washing, and PBS washing once again;
3) discard a supernatant and resuspend the cells with saline, followed by centrifugation and washing twice.
4) discard the supernatant, resuspend cells with saline, count the number and viability of cells, and make a cell suspension.

Based on the same inventive concept, the invention also applies the cells induced by the method for umbilical cord mesenchymal stem cells to differentiate insulin-like cells in the preparation of preparations for the treatment or adjuvant treatment of diabetes.

The beneficial effects realized by using the technical solution of the invention are as follows:

The invention carries out reasonable culture of purified umbilical cord mesenchymal stem cells by adopting culture medium having different substances, provides suitable environmental conditions for different stages of differentiation of the umbilical cord mesenchymal stem cells, and can efficiently and stably induce them to differentiate into insulin-like cells, and the percentage of insulin-positive cells can reach 80%.

And the cell preparation prepared after induction of umbilical cord mesenchymal stem cells of the invention can be used for the treatment of clinical diabetic patients with high therapeutic efficacy.

### 4. BRIEF DESCRIPTION OF ACCOMPANY DRAWINGS

FIG. 1 is a growth diagram of P2 generation human umbilical cord mesenchymal stem cells cultured in a φ90 mm culture dish of Embodiment 1.

FIG. 2 is a growth diagram of P4 generation human umbilical cord mesenchymal stem cells cultured in a T25 culture bottle of Embodiment 1.

FIG. 3 shows stem cells morphology after 6 days of induction of Embodiment 1.

FIG. 4 shows stem cells morphology after 12 days of induction of Embodiment 1.

FIG. 5 shows a flow detection diagram of stem cells of Embodiment 1.

FIG. 6 shows a multilineage differentiation staining of stem cells of the Embodiments.

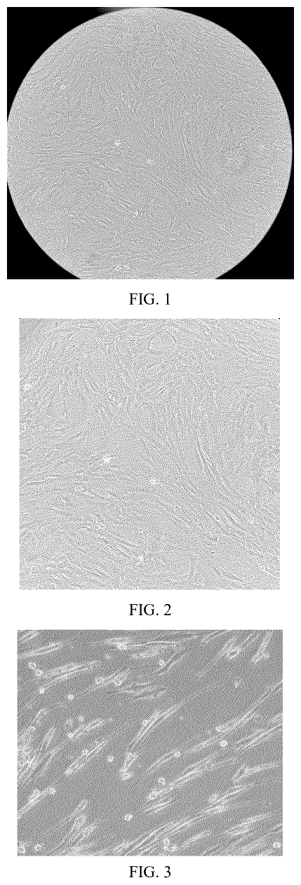

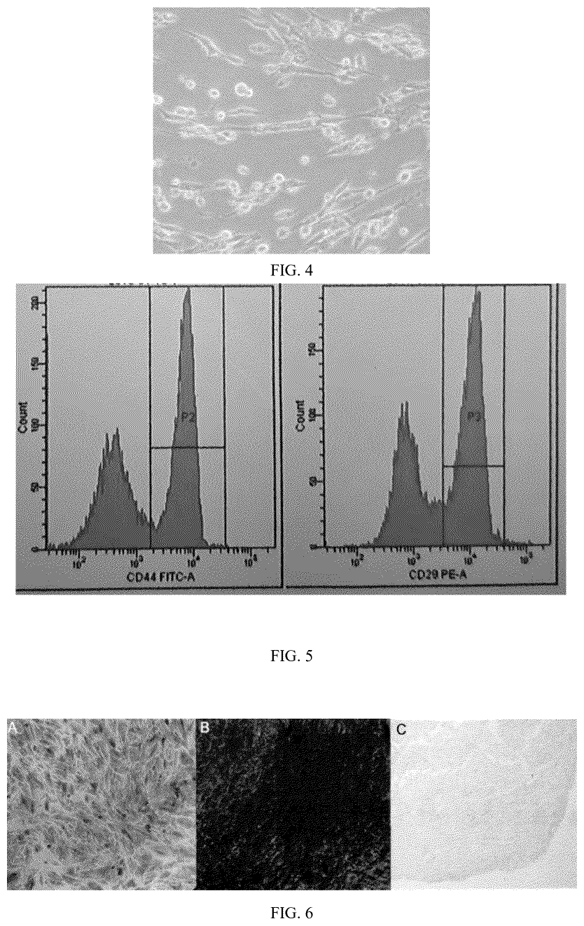

### 5. SPECIFIC EMBODIMENT OF THE INVENTION

In order to make the objects, technical solutions and advantages of the embodiments of the present invention clearer, the technical solutions in the embodiments of the invention will be clearly and completely described below with reference to the drawings in the embodiments of the invention. It should be understood that the specific embodiments described herein are for the sole purpose of explaining the invention and are not intended to limit the invention.

The technical solution of the invention is: a method of inducing umbilical cord mesenchymal stem cells to differentiate insulin-like cells, comprising the following steps:
S1: preparing and obtaining umbilical cord mesenchymal stem cells from umbilical cord;
preparation of umbilical cord mesenchymal stem cells in step S1 comprises the following steps:
S1.1 taking umbilical cord, removing arteries and veins, cutting it into small segments, affixing it to a culture dish, and adding a special medium for umbilical cord mesenchymal stem cells dropwise to the small segments of umbilical cord;
S1.2 after incubation for 2-6 hours, replenish the special medium for umbilical cord mesenchymal stem cells;
the special medium for umbilical cord mesenchymal stem cells of steps S1.1, S1.2, and S1.3 is α-MEM solution containing 5 wt % human platelet lysate, 100 U/ml penicillin, and 100 U/ml streptomycin.
S1.3 after continuing to culture for 5-7 days, digest and passage by using trypsin, and replenish the special medium for umbilical cord mesenchymal stem cells in the process of culture;
S1.4 when the cell density around tissue block reaches about 90%, trypsin digest cells, count them and transfer them for culture, and label them as P1 generation cells;
S1.5 perform cells passage until a 3rd generation, digest and collect the cells;
S1.6 purify and expand the 3rd generation cells collected at step S1.5 in α-MEM solution containing 5 wt % human platelet lysate.
S2: taking purified umbilical cord mesenchymal stem cells and add them to an α-MEM culture solution containing 10 ng/ml bFGF, 10 ng/ml EGF, and 5 wt % human platelet lysate, inducing differentiation of umbilical cord mesenchymal stem cells to nestin-positive progenitor cells NIP; after the cells are fully spread in the culture bottle, P4 generation cells are digested, and α-MEM culture solution containing 25 mM glucose and 5 wt % human platelet lysate is added, and cultured for 3 days at 37° C. in a 5% CO₂ cell culture incubator, and changes of the cells are observed dynamically;

In this step, mesenchymal stem cells purified from generation 3-6 are used for inducing differentiation, because the genome of mesenchymal stem cells from generation 1-2 had unstable factors and the number of cell expansion is insufficient, which is not suitable for clinical application, and after generation P7, the proliferative capacity and genetic stability of mesenchymal stem cells decrease dramatically, and the number of apoptosis increases, which is also not suitable for efficiently inducing differentiation of insulin cells.
S3: digesting the cells after the cells are fully spread in the culture bottle, adding to an α-MEM culture solution containing 25 mM glucose and 5 wt % human platelet lysate, at a temperature of 37° C.-38 ° C., and culturing with 5% CO₂ for 2-4 days;
S4: centrifugation, discard the culture supernatant, take cells, wash them, add them to an α-MEM culture solution containing 1 wt % ITS-A and 5 wt % human platelet lysate, at a temperature of 37° C.-38° C., and culturing with 5% CO₂ for 3-5 days;
the addition of ITS-A to the culture medium in this and the following steps can better promote cell proliferation, facilitate cell growth and differentiation in vitro, and improve cell activity and differentiation efficiency.
S5: take cells, discard culture supernatant, add them to an α-MEM culture solution containing 5 wt % human platelet lysate having 1 wt % ITS-A and 10 nM nicotinamide, at a temperature of 37° C.-38° C., 5% CO₂, culture for 2-4 days without changing the fluid during the period;
the addition of nicotinamide to the culture medium in this step and its following steps helps the differentiation and expansion of mesenchymal stem cells to insulin-like cells, and facilitates the increase of the differentiation and positivity rate of insulin cells.
S6: take cells, discard culture supernatant, add them to an α-MEM culture solution containing 5 wt % human platelet lysate having 30 ng/ml β-cell regulator, 10 ng/ml IGF, 1 wt % ITS-A and 10 nM nicotinamide, and culture for 2-5 days without changing the fluid during the period.

### Embodiment 1

This embodiment is further detailed for the implementation of the invention.
(1) Preparation of umbilical cord mesenchymal stem cells comprises the following steps:
1) take a fresh (healthy newborn baby delivered naturally or by cesarean section) umbilical cord, wash it with PBS to remove residual blood on surface of the umbilical cord in a GMP standardized laboratory, and cut it into segments of about 2 cm;
after washing again with PBS, two arteries in the umbilical cord were removed with ophthalmic forceps. The umbilical cord was cut along an umbilical vein and the vein was scraped off with ophthalmic forceps. The umbilical cord fragment with arteries and veins removed was washed and cut into about 1 mm³ size with ophthalmic forceps, and evenly affixed to a φ90 mm culture dish at about 5 mm intervals. After being placed for 5-10 minutes, α-MEM culture solution containing 10% autologous umbilical cord blood inactivated plasma, 100 U/ml penicillin, and 100 U/ml streptomycin was added dropwise to each tissue block (to just infiltrate the tissue block).

The umbilical cord donors should at least exclude diseases caused by pathogenic microbial infections such as hepatitis B virus, hepatitis C virus, HIV, syphilis spirochete, EB virus, cytomegalovirus, and so on.
2) After being placed in 37° C., 5% CO₂ incubator for 4 hours of incubation, remove and replenish with 2-3 ml of special culture medium for umbilical cord mesenchymal stem cells.

Semi-quantitatively replace the fresh medium (special medium for umbilical cord mesenchymal stem cells) every 2-3 days; the whole process should be gentle to avoid shedding of the tissue block.
3) After culturing about 5 days of umbilical cord tissue, some cells can be seen crawling out from around the tissue block in a form of tiny shuttle shape;
after 7 days, the cells began to proliferate rapidly, forming cell colonies of varying sizes, and when the cells were full grown, the cells were digested and passage with 0.25% trypsin.
at 12-15 days, a large number of long shuttle-shaped cells were visible in the culture dish climbing out of the tissue block.

When the cell density around the tissue block reached about 90%, trypsin digested the cells, counted and transferred the cells to 25 cm² cell culture bottles for culture at 1×10³/cm² and labeled as P1 generation cells.

After the cells were fully spread in the culture bottle (about 3 days), 1:3 normal passage. And observe the cell growth condition at any time.

When the cells were passaged to the 3rd generation, trypsin digestion was performed and a certain amount of cells were collected for cell counting, viability and phenotype determination and frozen storage.

Adherent cells all expressed CD44, CD29, low expression of CD106, did not express hematopoietic cell phenotypes CD14, CD34, CD45 and endothelial cell phenotype CD31, and did not express HLA-DR: the cell doubling time was 30 h, and the cell cycle analysis showed that the percentage of cultured mesenchymal stem cells in G0-G1 phase and +G2+M phase were 78.84% and 11.16%.
4) The 3rd generation cells were purified and expanded in α-MEM medium containing 5 wt % human platelet lysate, 100 U/ml penicillin and 100 U/ml streptomycin;
in the process of mesenchymal stem cells culture, the cells will be purified gradually as the number of generations increases. P3 generation can be seen, and the cells are growing in a long shuttle shape close to the wall, and distributed in a typical “fish school-like” or “swirl-like” manner.
(2) Inducing differentiation of insulin-like cells includes the following steps:
1) take purified P3 generation cells and induce differentiation of MSC to nestin positive progenitor cells (NIP) with factors such as bFGF and EGF;
2) On day 4, discard cell cultural supernatant, washed once with PBS, and added with an α-MEM culture solution containing ITS-A (insulin-transferrin-selenium-sodium pyruvate) and 5 wt % human platelet lysate: continue to culture for 4 days at 37° C. in a 5% CO₂ cell incubator without changing the fluid, and cells status was observed every day.
3) On day 8, discard cell cultural supernatant, and added with an α-MEM culture solution containing 5 wt % human platelet lysate having 1 wt % ITS-A and 10 mM nicotinamide, culture at 37° C. in a 5% CO₂ cell culture incubator for 3 days without changing the fluid, and the cell changes were observed dynamically.
4) On the 11th day, discard cell cultural supernatant, add an α-MEM culture solution containing 5 wt % human platelet lysate having 30 ng/ml β-cell regulator, 10 ng/ml IGF, 1 wt % ITS-A and 10 nM nicotinamide, and culture at 37° C., 5% CO₂ cell culture incubator for 4 days without changing the fluid during the period, and observe the cell changes dynamically, and then carry out identification from various aspects such as morphology.

During the process of inducing differentiation of insulin-like cells, the cells gradually became polygonal and rounded from shuttle shape, and the refractive property was enhanced until most of the cells became round.

Flow cytometry identification of human umbilical cord MSCs after induced differentiation:

Take the induced cells, perform PBS washing and trypsin digestion (digestion time should not be too long, usually 20 seconds is sufficient), add them to a culture medium and added with trypsin, placed into a 15 ml centrifugal tube, followed by placing them in a centrifugal machine for centrifugation (1000 rpm/5 min), and washed with PBS once again.

After cell counting, the cells (5×10⁵) were dispensed into 15 ml centrifuge tubes, centrifuged and volume was fixed with PBS containing 0.1 wt % FBS to 400 μl. Subsequently, relative dose of antibody was added according to recommended dosage in antibody instruction manual, and incubated for 30 min at 4° C. away from light.

At the end of staining, performing PBS washing and subsequently fix the volume to 600 μl and the cells were filtered through a 200-mesh nylon gauze into a flow tube.

On-machine detection (FACSCalibur), eliminating non-specific signal interference to ensure accuracy of the experiment, taking an antibody tube as a negative reference, and detecting corresponding antibody expression quantity of each tube after adjustment, wherein the result is shown in FIG. 5. The percentage of insulin positive cells can reach 80%.

### Embodiment 2

The cell preparation used in this embodiment was prepared by removing supernatant from cells cultured in step 4) of Embodiment 1.

Take the induced cells, perform PBS washing and trypsin digestion (digestion time should not be too long, usually 20 seconds is sufficient), add them to a culture medium (α-MEM) and added with trypsin, placed into a 15 ml centrifugal tube, followed by placing them in a centrifugal machine for centrifugation (1000 rpm/5 min), and washed with PBS once again.

Discard a supernatant and resuspend the cells with saline, followed by centrifugation of centrifugal machine and washing twice.

Discard the supernatant, resuspend cells with saline, count the number and viability of cells, and make a cell suspension.

A total of 325 patients (203 males, 122 females, age group 10-70 years, 129 patients with type 1 diabetes mellitus and 196 patients with type 2 diabetes mellitus) were recruited for clinical diagnosis of diabetes mellitus, all of whom, were evaluated by endocrinology consultation. All patients were treated with dietary control and appropriate physical activity.

Before treatment with the cell preparation, insulin and oral hypoglycemic drugs were adjusted to maintain the patients' blood glucose at a stable level (blood glucose level at 7.0-10.0 mmol/L, glycated hemoglobin level at 6.0-8.0%), and the cell preparation is intravenously infused for 1 week, and then once every 2 weeks for a total of 2-3 times, with the total number of cells inputted in each patient ranging from (1-3)×10⁶/kg, and the average total number of cells inputted in per patient was 1.8×10⁶/kg.

No hypoglycemic drugs or insulin injections were taken during the treatment with cell preparations, unless the patients had obvious hypoglycemia or hyperglycemia, which required urgent treatment.

### Criteria for Evaluation of Therapeutic Efficacy

(1) Significant effect: without taking oral hypoglycemic drugs or injecting insulin, blood glucose is reduced by more than 20%.
(2) Effective: the dosage of hypoglycemic drugs or insulin is reduced by ⅓ under the premise that blood glucose is controlled at the pre-treatment level;

All patients were subjected to a detailed medical history, physical examination and relevant laboratory tests:
(1) Medical history; age, gender, height, weight, duration of diabetes mellitus, dosage of insulin and oral hypoglycemic drugs, glycemic control, history of drug and food allergy;
(2) Physical examination: presence of diabetic complications;
(3) Examination:
\textcircled{1} Fasting and 2 h blood glucose after three meals;
\textcircled{2} C-peptide release test; each patient was subjected to a venous blood test plus blood glucose and C-peptide on an empty stomach, and then orally take 100 g of steamed bun or bread, timing from the starting to eat, extract blood glucose and C-peptide at 1, 2 and 3 h;
\textcircled{3} HbA1c;
\textcircled{4} T cell subsets;
\textcircled{5} Regulate T lymphocytes
\textcircled{6} any obvious adverse reactions, if there are adverse reactions, they should be recorded and given positive symptomatic treatment.

### Evaluating the Treatment Effect

through 325 patients clinically, the cell preparation containing the insulin-like cells prepared by the invention is used for treatment, and the result shows that the blood glucose level of 293 patients is reduced by 20 percent and the glycosylated hemoglobin is recovered to be normal under the premise that no oral hypoglycemic drugs or insulin injection is taken; the blood sugar level is stable, and no complication occurs; glycosylated hemoglobin
98 patients with type 2 diabetes mellitus did not take hypoglycemic drugs or take reduced dosage of drugs, and it was observed that no renal function and peripheral nerve complications occurs. Blood glucose and glycosylated hemoglobin fluctuate less. Finally, the purpose of controlling the occurrence and development of diabetic complications and achieving clinical cure is achieved clinically.

In addition, in the process of tissue repair, the cell preparation of the invention can protect residual β-cells through paracrine action, improve the insulin sensitivity of peripheral target tissues, inhibit oxidative stress, stimulate generation of capillaries, promote cell proliferation and reduce apoptosis. The mesenchymal stem cells can promote the regeneration of pancreatic β cells: has the potential of differentiating to pancreatic β cells, and can replenish pancreatic β cell loss caused by diabetes, thereby restoring or improving insulin secretion capacity. 2. Immunoregulation effect: in type 1 diabetes, the autoimmune response is the leading cause of pancreatic β cell destruction. The mesenchymal stem cells can relieve autoimmune reaction and protect residual pancreatic β cells by inhibiting T cell proliferation, reducing inflammatory factor secretion and the like. 3. Improving microangiopathy: diabetes patients often have microangiopathy affecting organ function. The mesenchymal stem cells can promote the formation of new vessels, improve blood circulation and help to relieve complications caused by diabetes. 4. Anti-inflammatory function: the mesenchymal stem cells have obvious anti-inflammatory effect, can reduce the generation of inflammatory mediators, improve the insulin resistance and have positive significance for treating type 2 diabetes.

The foregoing is only preferred embodiments of the invention, and it should be noted that for a person of ordinary skill in the art, a plurality of improvements and embellishments may be made without departing from principles of the invention, and these improvements and embellishments should also be considered as the protection scope of the invention.

1. A method of inducing umbilical cord mesenchymal stem cells to differentiate insulin-like cells, comprising the following steps:
S1: preparing and obtaining umbilical cord mesenchymal stem cells from umbilical cord;
S2: taking purified umbilical cord mesenchymal stem cells and add them to an α-MEM culture solution containing bFGF, EGF, and 5 wt % human platelet lysate, inducing differentiation of umbilical cord mesenchymal stem cells to nestin-positive progenitor cells NIP;
S3: digesting the cells after the cells are fully spread in the culture bottle, adding an α-MEM culture solution containing glucose and 5 wt % human platelet lysate, and culturing for 2-4 days;
S4: take cells, discard the culture supernatant, add them to an α-MEM culture solution containing ITS-A and 5 wt % human platelet lysate, culture for 3-5 days;
S5: take cells, discard the culture supernatant, add them to an α-MEM culture solution containing 5 wt % human platelet lysate having ITS-A and nicotinamide, and culture for 2-4 days without changing the fluid during the period;
S6: take cells, discard the culture supernatant, add them to an α-MEM culture solution containing 5 wt % human platelet lysate having β-cell regulator, IGF, ITS-A and nicotinamide, and culture for 2-5 days without changing the fluid during the period.

2. The method of inducing umbilical cord mesenchymal stem cells to differentiate insulin-like cells of claim 1, wherein concentration of glucose in the α-MEM culture solution in step S3 is 25 mM.

3. The method of inducing umbilical cord mesenchymal stem cells to differentiate insulin-like cells of claim 1, wherein concentration of ITS-A in the α-MEM culture solution in steps S4 to S6 is 1 wt %.

4. The method of inducing umbilical cord mesenchymal stem cells to differentiate insulin-like cells of claim 1, wherein concentration of nicotinamide in the α-MEM culture solution in each of steps S5 and S6 is 10 mM.

5. The method of inducing umbilical cord mesenchymal stem cells to differentiate insulin-like cells of claim 1, wherein conditions of cell culture in steps S3 to S6 are all: temperature: 37° C.-38° C., 5% CO₂.

6. The method of inducing umbilical cord mesenchymal stem cells to differentiate insulin-like cells of claim 1, wherein preparation of umbilical cord mesenchymal stem cells in step S1 comprises the following steps:
S1.1 taking umbilical cord, removing arteries and veins, cutting it into small segments, affixing it to a culture dish, and adding a special medium for umbilical cord mesenchymal stem cells dropwise to the small segments of umbilical cord;
S1.2 after incubation for 2-6 hours, replenish the special medium for umbilical cord mesenchymal stem cells;
S1.3 after continuing to culture for 5-7 days, digest and passage by using trypsin, and replenish the special medium for umbilical cord mesenchymal stem cells in the process of culture;
S1.4 when the cell density around tissue block reaches about 90%, trypsin digest cells, count them and transfer them for culture, and label them as P1 generation cells;
S1.5 perform cells passage until a 3rd generation, digest and collect the cells;
S1.6 purify and expand the 3rd generation cells collected at step S1.5 in α-MEM solution containing 5 wt % human platelet lysate.

7. The method of inducing umbilical cord mesenchymal stem cells to differentiate insulin-like cells of claim 6, wherein the special medium for umbilical cord mesenchymal stem cells of steps S1.2 is α-MEM solution containing 5 wt % human platelet lysate, 100 U/ml penicillin, and 100 U/ml streptomycin.

8. A cell preparation, wherein, the cell preparation is prepared from cells induced by the method for inducing the umbilical cord mesenchymal stem cells to differentiate insulin-like cells from claim 1.

9. A preparation method of the cell preparation, which is characterized by comprising the following steps:
1) taking cells induced by the method for inducing the umbilical cord mesenchymal stem cells to differentiate insulin-like cells from claim 1;
2) centrifuging the induced cells after trypsin digestion using PBS washing, and PBS washing once again;
3) discard a supernatant and resuspend the cells with saline, followed by centrifugation and washing twice;
4) discard the supernatant, resuspend cells with saline, count the number and viability of cells, and make a cell suspension.

10. An application of the cells induced by the method for umbilical cord mesenchymal stem cells to differentiate insulin-like cells from claim 1 in the preparation of preparations for the treatment or adjuvant treatment of diabetes.
